(19) United States
(12) Patent Application Publication
(10) Pub. No.: US 2005/0267087 A1
(43) Pub. Date: Dec. 1, 2005

(54) Inflammatory eye disease

(76) Inventors: Vassiliki Poulaki, Roslindale, MA (US); Joan W. Miller, Winchester, MA (US)

Correspondence Address: FISH & RICHARDSON PC, P.O. BOX 1022, MINNEAPOLIS, MN, 55440-1022, US

(21) Appl. No.: 11/115,687

(22) Filed: Apr. 27, 2005

Related U.S. Application Data
(60) Provisional application No. 60/566,493, filed on Apr. 28, 2004.

(51) Int. Cl. A61K 31/47

(52) U.S. Cl. 514/183

(57) ABSTRACT

The present invention provides methods, kits and compositions for treating uveitis in a subject using Hsp90 inhibitors.

### CLAIM OF PRIORITY

This application claims priority under 35 USC § 119(e) to U.S. Provisional Patent Application Ser. No. 60/566,493, filed on Apr. 28, 2004, the entire contents of which are hereby incorporated by reference.

### TECHNICAL FIELD

This invention relates to methods of treating inflammatory eye disease.

### BACKGROUND

Uveitis is one of the leading causes of blindness in the world (Nussenblatt, Int. Ophthalmol. 14:303-308 (1990)). It has been estimated that uveitis accounts for 10-15% of all cases of total blindness in the USA, with the majority of patients of working age (20-50 years old). Severe vision-threatening complications include cystoid macular edema, secondary glaucoma, secondary cataract, vitreous opacities, and retinal scars (Nussenblatt et al., Uveitis, Fundamentals and Clinical Practice, 2nd ed. (Mosby, St. Louis, 1996)).

The etiopathogenesis of this group of diseases is largely unknown, but disturbances of immune mechanisms have been hypothesized to play a central role, and immunological abnormalities have been detected in many uveitis patients. In cases of endogenous uveitis where no infectious etiology can be identified, immunopathological findings from enucleated eyes point to autoimmune inflammatory responses as a cause (Bloch-Michel and Nussenblatt, Am. J. Ophthalmol. 103(2):234-5 (1987)). The points at which the inflammatory response may be successfully inhibited generally depend on whether the immune response was directed against host or foreign antigen in the tissue. When no overt infectious or neoplastic etiology is found, treatment may be directed towards dampening the resulting inflammatory cascade and hopefully reducing tissue damage.

### SUMMARY

The invention described herein is based, in part, on the discovery that administration of an inhibitor of Heat shock protein 90 (Hsp90), e.g., geldanamycin, in a rat model of endotoxin-induced uveitis (EIU) decreases leukocyte infiltration of the retinal tissue, decreases Vascular Endothelial Growth Factor (VEGF), Nuclear Factor Kappa B (NF-κB), and Tumor Necrosis Factor alpha (TNF-α) levels, and consequently reduces the breakdown of the blood-retinal barrier that is a common result of uveitis.

Thus, the invention provides methods for treating uveitis in a subject, by administering to the subject a therapeutically effective amount of an inhibitor of Hsp90. As used herein, “treating” includes any therapy that is administered either after the disease is diagnosed, or before the subject is diagnosed in a subject at risk for getting uveitis, that can, e.g., ameliorate a symptom of, prevent, and/or delay the development or progression of, uveitis. Risk factors for development of uveitis include a history of an autoimmune disease, infection, or toxin exposure. Uveitis in one eye may be a risk factor for development of uveitis in the other eye.

The invention also provides therapeutic compositions that include one or more inhibitors of Hsp90 and a carrier, and are specially formulated for administration in the eye. Also included are kits including the therapeutic compositions and instructions for administering the inhibitor to an individual to treat uveitis.

An “inhibitor of Hsp90” (also referred to herein as a “Hsp90 inhibitor”) is a compound that disrupts the structure and/or function of an Hsp90 chaperone protein and/or a protein that is dependent on Hsp90. In some embodiments, an Hsp90 inhibitor can be a member of the ansamycin family, e.g., an ansamycin antibiotic such as geldanamycin or a geldanamycin analog such as 17-Allylamino-17-demethoxygeldanamycin (17AAG); a macbecin such as macbecin I or macbecin II or an analog thereof; herbimycin or an analog thereof, radicicol or a radicicol analog; or a derivative or analog thereof.

In some embodiments, the inhibitor of Hsp90 is administered intravenously, orally, intravitreally, transclerally, subtenons, subcutaneously, or topically.

In some embodiments, the inhibitor of Hsp90 is administered in conjunction with a second therapeutic modality, e.g., systemic or local steroid therapy, or an immune suppressive therapy. In some embodiments, the inhibitor of Hsp90 is administered in conjunction with a cycloplegic or mydriatic agent.

In some embodiments, the inhibitor of Hsp90 is in a pharmaceutical composition further comprising a carrier.

As used herein, “uveitis” refers to inflammation within the eye. The uvea includes the iris at the front of the eye, the ciliary body, and the choroid toward the back of the eye. Uveitis includes, but is not limited to, iritis (inflammation of the iris), cyclitis (inflammation of the ciliary body) anterior uveitis or iridocyclitis (both the iris and the ciliary body are involved), choroiditis or posterior uveitis (inflammation of the choroid), chorioretinitis (the retina is inflamed adjacent to the choroids), panuveitis (inflammation of the iris, ciliary body and choroid), and pars planitis (the inflammation is especially pronounced at the pars plana, an area just behind the ciliary body).

As used herein, a “geldanamycin analog” is an antineoplastic antibiotic drug that belongs to the family of drugs called ansamycins. The ansamycins cause disruption of Hsp90-client protein complexes and lead to proteosome-mediated degradation of client proteins. Exemplary analogs include macbecin I (see, e.g., Ono et al., Gann. 73(6):938-44 (1982); Tanida et al., J. Antibiot. (Tokyo) 33(2):199-204 (1980)) and 17-Allylamino-17-demethoxygeldanamycin (17AAG).

As used herein, a “derivative” of a parent compound is a compound that is structurally related to the parent compound, and retains Hsp90-binding and inhibition activity. Suitable derivatives can be prepared through chemical manipulation and/or genetic engineering. Compounds having improved solubility properties and compounds having conformations optimized to bind Hsp90 are also provided. An “analog” is structural derivative of a parent compound that differs from it by a single element.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

### DESCRIPTION OF DRAWINGS

FIG. 1 is a Western blot showing that geldanamycin treatment does not affect total CD14 expression in leukocytes in rats with endotoxin-induced uveitis (ER;). LPS, lipopolysaccharide; DMSO, dimethyl sulfoxide (control); GA, geldanamycin.

FIG. 2 is a Western blot showing that geldanamycin treatment decreases expression of the membrane form of CD14 in leukocytes in rats with EIU.

FIG. 3 is a bar graph illustrating that geldanamycin treatment decreases LPS-induced upregulation of retinal hypoxia-inducible factor 1, alpha subunit (HIF-1a) levels in rats with EIU.

FIG. 4 is a bar graph illustrating that geldanamycin treatment decreases LPS-induced upregulation of retinal Nuclear Factor kappa B (NF-κB) levels in rats with EIU.

FIG. 5 is a bar graph illustrating that geldanamycin treatment decreases LPS-induced upregulation of Vascular Endothelial Growth Factor (VEGF) levels in rats with EIU.

FIG. 6 is a bar graph illustrating that geldanamycin treatment decreases lipopolysaccharide (LPS)-induced upregulation of Tumor Necrosis Factor alpha (TNF-α) levels in rats with EIU.

FIG. 7 is a bar graph illustrating that geldanamycin reduces myeloperoxidase activity in leukocytes from rats with EIU. MPO, myeloperoxidase.

FIG. 8 is a bar graph illustrating that geldanamycin treatment decreases LPS-induced upregulation of retinal intercellular adhesion molecule 1 (ICAM-1) levels in rats with EIU.

FIGS. 9-11 are bar graphs illustrating the effect of geldanamycin treatment on leukocyte adhesion in rats with EIU in both arteries and veins (FIG. 9); veins (FIG. 10); or arteries (FIG. 11). GA, geldanamycin.

FIG. 12 is a bar graph illustrating that geldanamycin decreases blood-retinal barrier breakdown in EIU. GA, geldanamycin.

### DETAILED DESCRIPTION

Described herein are methods for treating uveitis using Hsp90 inhibitors, as well as compositions including such inhibitors that are specially formulated for use in the eye.

Uveitis

Uveitis, or inflammation of the uvea, includes, but is not limited to, uveitis associated with the diseases listed in Table 1. Uveitis may cause vague clinical signs that may include blinking, squinting, watery discharge from the eye, and/or light sensitivity (photophobia), without any obvious changes to the eye itself. The cornea, which is normally clear, may appear dull or hazy blue due to uveitis. In some cases, the cornea becomes cloudy due to white blood cells accumulating on the inside of the cornea. The conjunctiva may become red and swollen. In some cases of uveitis, the iris may become red or change color. Uveitis is typically diagnosed by an examination of structures of the eye. In more advanced cases, changes are visible without magnification.

TABLE 1
Causes of Uveitis

| | |
|---|---|
| Infectious diseases | Suspected immune-mediated diseases |
| Viral | Ankylosing spondylitis |
| Human immunode- | Behcet's disease |
| ficiency virus-I | |
| Herpes simplex | Crohn's disease |
| Herpes zoster | Drug or hypersensitivity reaction |
| Cytomegalovirus | Interstitial nephritis |
| Bacterial or spirochetal | Juvenile rheumatoid arthritis |
| Tuberculosis | Kawasaki's disease |
| Leprosy | Multiple sclerosis |
| Proprionobacterium | Psoriatic arthritis |
| Syphilis | Reiter's syndrome |
| Whipple's disease | Relapsing polychondritis |
| Leptospirosis | Sarcoidosis |
| Brucellosis | Sjogren's syndrome |
| Lyme disease | Systemic lupus erythematosus |
| Parasitic (protozoan | Ulcerative colitis |
| or helminthic) | |
| Toxoplasmosis | Vasculitis |
| Acanthameba | Vitiligo |
| Toxocariasis | Vogt Koyanagi Harada Syndrome |
| Cysticercosis | |
| Onchocerciasis | |
| Fungal | |
| Histoplasmosis | |
| Coccidioidomycosis | |
| Candidiasis | |
| Aspergillosis | |
| Sporotrichosis | |
| Blastomycosis | |
| Cryptococcosis | |
| Masquerade syndromes | Syndromes confined primarily to the eye |
| Leukemia | Acute multifocal placoid pigmentary |
| Lymphoma | epitheliopathy |
| Retinitis pigmentosa | Acute retinal necrosis |
| Retinoblastoma | Birdshot choroidopathy |
| | Fuch's heterochromic cyclitis |
| | Glaucomatocyclitic crisis |
| | Lens-induced uveitis |
| | Multifocal choroiditis |
| | Pars planitis |
| | Serpiginous choroiditis |
| | Sympathetic ophthalmia |
| | Trauma |

Uveitis can include, but is not limited to, acute anterior uveitis, e.g., associated with ankylosing spondylitis, Reiter's syndrome, herpes zoster ophthalmicus or sarcoidosis; chronic uveitis, e.g., from Still's disease, or Fuch's heterochromic iridocyclitis; intermediate uveitis, e.g., from Whipple's disease or multiple sclerosis; posterior uveitis, e.g., associated with Bechet's disease, AIDS, CMV, toxoplasmosis, cryptococcidomycosis, secondary syphilis, atypical mycobacteria, toxocara, tuberculosis, or acute retinal necrosis; retinal pigment epitheliopathies (e.g., acute multifocal placoid epitheliopathy); Vogt-Koyanagi-Harada syndrome; sympathetic opthalmitis; birdshot chorioretinopathy; serpiginous or geographic choroiditis; phaco-anaphylactic uveitis; Stevens Johnson pseudomembranous conjunctivitis; allergic conjunctivitis; ischemic optic neuropathy due to temporal arteritis; episcleritis; scleritis; papillitis; pterygium; systemic lupus erythematosus; and trachoma.

Current therapeutic algorithms for uveitis include the use of either local or systemic corticosteroids. Long term steroid therapy has various side effects such as increased intraocular pressure in “steroid responders,” and cataract formation from local therapy, to serious side effects from systemic therapy (severe infections, hyperglycemia, edema, osteonecrosis, myopathy, peptic ulcer disease, hypokalemia, osteoporosis, euphoria, psychosis, myasthenia gravis, and growth suppression).

In combination with topical or systemic corticosteroids, current treatment methodologies often include the administration of cycloplegics, which paralyze the ciliary muscles and cause dilatation of the pupil, useful in providing pain relief. In patients with severe cases of uveitis who are unresponsive to steroids or in those patients with complications associated with the usual therapy, immunosuppressants can be used, such as cyclosporin A or azathioprine (Rosenbaum and George, Current Ocular Therapy 5:519-21 (2000)). Although present treatments are successful in some cases, success is often limited by the required long term use, resistance in some patients, and significant side effects (Id.).

In general, the methods described herein include identifying a subject with uveitis, and administering a therapeutically effective amount of an Hsp90 inhibitor to the subject. In some embodiments, the administration is ocular, e.g., for application into or around the eye, e.g., by injection into the eye, or by eye drops. In some embodiments, the methods include co-administration of an Hsp90 inhibitor with a conventional treatment, e.g., a steroid.

The experiments described herein utilized geldanamycin in a rat model of endotoxin-induced uveitis (EIU) and found that it decreased leukocyte infiltration of the retinal tissue, it decreased NF-κB, VEGF and TNF-α levels and consequently reduced the breakdown of the blood-retinal barrier.

Geldanamycin (GA), a benzoquinone ansamycin antibiotic, is a natural inhibitor of Hsp90, a chaperone molecule that interacts with a variety of intracellular client proteins to facilitate their proper folding, prevent misfolding and preserve their 3-dimensional conformation in a functionally competent state (Isaacs et al., Cancer Cell 3:213-7 (2003)). Through its inhibitory effect on Hsp90 activity, geldanamycin affects several key growth factor-initiated signal transduction pathways, including suppression of cell surface receptors, induction of misfolding and subsequent proteasomal degradation and depletion of crucial kinases and transcription factors (Goetz et al., Ann Oncol. 14(8):1169-76 (2003).

Geldanamycin represents a novel anti-inflammatory compound, that targets multiple intracellular pathways important for cellular immune response and inhibits the expression of inflammatory factors as TNF-alpha. It has also been shown that geldanamycin suppresses cardinal manifestations of autoimmune encephalomyelitis in an animal model by inhibiting key inflammatory mediators such as nitric oxide, and attenuates the oxidative injury in hemorrhagic shock by restoring a defective inflammatory response in vivo (Murphy et al., J. Neurosci. Res. 67(4):461-70 (2002); Pittet et al., J. Physiol. 538(Pt 2):583-97 (2002); Poulaki et al., Am J Pathol. 165(2):457-69 (2004)).

The results described herein demonstrate that 17-Allylamino-17-demethoxygeldanamycin (17AAG), a semi-synthetic analog of geldanamycin, reduces the expression of vascular endothelial growth factor (VEGF), which plays a crucial role in the pathogenesis of uveitis. Administration of the parental compound, geldanamycin, exerts potent anti-inflammatory effects in animal models of autoimmune encephalomyelitis, toxic shock syndrome and portal hypertensions (Murphy et al., J. Neurosci. Res. 67(4):461-70 (2002); Pittet et al., J. Physiol. 538(Pt 2):583-97 (2002); Winklhofer et al., J. Biol. Chem. 276(48):45160-7 (2001)). 17AAG is in Phase II trials for various neoplasias and has demonstrated excellent efficacy below the maximum tolerable dose (Maloney and Workman, Expert Opin. Biol. Ther. 2(1):3-24 (2002)).

17AAG is a small molecule that can be administered intravenously, orally, intravitreally, transclerally, subtenons, subcutaneously, or topically, e.g., as an ointment. Also, it can be encapsulated in microspheres or liposomes or placed in a device for longer release, e.g., an ocular implant. As shown herein, 17AAG reduces the expression of permeability factors as VEGF and TNF-α; these factors may cause the ocular damage consistent with uveitis (Rosenbaum and George, Uveitis. Current Ocular Therapy 5:519-21 (2000)). Phase I trials in various neoplasias showed that 17AAG has a favorable pharmacokinetic profile with transient and manageable side effects (Maloney and Workman, Expert Opin. Biol. Ther. 2(1):3-24 (2002)). Therefore, geldanamycin and other Hsp90 inhibitors represent a potential safe and effective treatments for uveitis.

Hsp90 Inhibitors

The eukaryotic heat shock protein 90s (Hsp90s) are ubiquitous chaperone proteins that bind and hydrolyze ATP. Hsp90s are believed to be involved in folding, activation and assembly of a number of client proteins, including proteins involved in signal transduction, cell cycle control, and transcriptional regulation.

Hsp90 proteins are highly conserved in nature, and include Hsp90 alpha and beta, Grp94, and Trap-1. For exemplary protein sequences see, e.g., NCBI accession Nos. NP_{—}005339.2 and NP_{—}001014390.1 (Homo sapiens alpha and beta Hsp90, respectively); P07901 (Mus musculus); NP_{—}001004082.2, AAT99568.1 (Rattus norvegicus); AAA36992.1 (Cricetulus griseus); JC1468 and HHCH90 (Gallus gallus); AAF69019.1 (Sarcophaga crassipalpis); AAC21566.1 (Danio rerio), AAD30275.1 (Salmo salar), NP_{—}999138.1 (Sus scrofa), NP_{—}015084.1 (Saccharomyces cerevisiae), and CAC29071 (frog).

The Hsp90 inhibitors can be specifically directed against an Hsp90 of the specific host patient, or can be identified based on reactivity against an Hsp90 homolog from a different species, or an Hsp90 variant. The inhibitors can be, for example, ring-structured antibiotics, e.g., benzoquinone ansamycins, or other types of molecules, e.g., antisense nucleic acids, or molecules such as radicicol and analogs thereof.

In vivo and in vitro studies indicate that without the aid of co-chaperones Hsp90is unable to fold or activate proteins. For steroid receptor conformation and association in vitro, Hsp90 requires Hsp70 and p60/Hop/Sti1 (Caplan, Trends in Cell Biol., 9:262-68, (1999)). In vivo Hsp90 may interact with Hsp70 and its co-chaperones. Other co-chaperones associated with Hsp90s in higher eukaryotes include Hip, Bag1, Hsp40/Hdj2/Hsj1, immunophilins, p23, and p50 (Caplan, (1999) supra). The binding of ansamycins to Hsp90 has been reported to inhibit protein refolding and to cause the proteasome dependent degradation of a select group of cellular proteins (Sepp-Lorenzino, et al., J. Biol. Chem., 270:16580-16587 (1995); Whitesell, et al., Proc. Natl. Acad. Sci. USA, 91: 8324-8328 (1994)).

In some embodiments, the Hsp90 inhibitors are small molecules. As used herein, “small molecules” refers to small organic or inorganic molecules of molecular weight below about 3,000 Daltons. In general, small molecules useful for the invention have a molecular weight of less than 5,000 Daltons (Da). Small molecule inhibitors based on purine and pyrazole scaffolds are known in the art, e.g., the purine-based small molecules CCT018159 and analogs thereof (Aherne et al., Proc. AACR 44, Abstract #4002 (2004); Wright et al., Chem. Biol. 11(6):775-85 (2004)).

A number of suitable Hsp90 inhibitors are known in the art, including compounds that bind to the ATP/ADP-binding pocket in the geldanamycin-binding domain of Hsp90that is highly conserved across species (residues 9-232, see Stebbins et al., Cell, 89:239-250 (1997); Schulte et al., Cell Stress Chaperones 3(2):100-8 (1998). This results in the depletion of Hsp90 client proteins, particularly kinases that are involved in signal transduction and oncogenesis (including c-Raf1, akt and cdk4) together with mutant p53. Two main classes of Hsp90 inhibitors are the benzoquinone ansamycin antibiotics, including benzoquinone ansamycin analogs such as herbimycin A, macbecin I, geldanamycin and 17-amino derivatives of geldanamycin, e.g., 17-(allylamino)-17-desmethoxygeldana-mycin (17AAG), and another natural product, radicicol.

Methods for identifying Hsp90 inhibitors, e.g., small molecule Hsp90 inhibitors, using target based screening, structure-based rational design, and high throughput screening, are known in the art. See, e.g., Neckers, Curr. Med. Chem. 10(9):733-9 (2003); Rowlands et al., Anal. Biochem. 327(2):176-83 (2004); Dymock et al., Exp. Op. Ther. Patents 14(6):837-847 (2004); Workman, Cancer Letters 206:149-157 (2004); Aherne et al., Methods. Mol. Med. 85:149-61 (2003); and Chiosis et al., Curr. Cancer Drug Targets 3(5):371-6 (2003).

Benzoquinone Ansamycin Antibiotics and Analogs

Geldanamycin and Geldanamycin Analogs

Geldanamycin (GA) is a benzoquinone ansamycin antibiotic produced by Streptomyces hygroscopicus that exhibits a potent antitumor activity. Geldanamycin binds specifically to heat shock protein 90 (Hsp90), leading to the destabilization and degradation of its client proteins (Whitesell et al., Proc. Natl. Acad. Sci. USA 91(18):8324-8 (1994)). Hsp90 acts as a molecular chaperone, and is critical for the folding, assembly and activity of multiple mutated and overexpressed signaling proteins that promote the growth and/or survival of tumor cells. Hsp90 client proteins destabilized by Geldanamycin and 17AAG include steroid receptors such as androgen and estrogen receptors; tyrosine kinases such as v-Src, Bcr-Ab1, erbB2; transcription factors such as p53, hypoxia-inducible factor 1a (HIF-1a), and EF-2; and serine/threonine kinases such as Raf-1, and Akt (Neckers, Trends Mol. Med. 8(4 Suppl):S55-61 (2002)).

Geldanamycin analogs suitable for use in the methods described herein include, but are not limited to, geldanamycin and 17-amino derivatives of geldanamycin, e.g., 17AAG (see formula I, below). 17AAG and analogs thereof are described in U.S. Ser. Nos. 10/212,962 and 10/461,194; see also Sasaki et al., U.S. Pat. No. 4,261,989 for methods of synthesis of 17AAG. Other 17-amino derivatives of geldanamycin include 17-(2-dimethylaminoethyl)amino-17-desmethoxy-geldanamycin (17-DMAG), see Snader et al., U.S. 2004/0053909 A1 (2004) for synthesis of 17-DMAG; 11-oxogeldanamycin, and 5,6-dihydrogeldanamycin, both disclosed in U.S. Pat. Nos. 4,261,989, 5,387,584 and 5,932,566; 11-O-methyl-17-(2-(1-azetidinyl)ethyl)amino-17-demethoxygeldanamycin (A), 11 -O-methyl-17-(2-dimethylaminoethyl)amino-17-demethoxygeldanamycin (B), and 11-O-methyl-17-(2-(1-pyrrolidinyl)ethyl)amino-17-demethoxygeldanamycin (C), described in U.S. Ser. No. 10/825,788, and PCT Application No. PCT/US04/11638; additional geldanamycin derivatives are described in Santi et al., U.S. 2003/0114450 A1 (2003).

| | | | | Formula I |
|---|---|---|---|---|
| 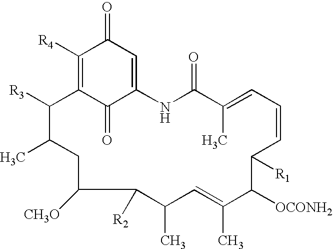 | | | | |
| | R₁ | R₂ | R₃ | R₄ |
| Herbimycin A | —OCH₃ | —OCH₃ | —OCH₃ | —H |
| Macbecin I | —CH₃ | —OCH₃ | —OCH₃ | —H |
| Geldanamycin | —OCH₃ | —OH | —H | —OCH₃ |

17AAG

17AAG (see Formula II) is a less toxic and more stable analog of geldanamycin (GA) (Schulte and Neckers, Cancer Chemother. Pharmacol. 42(4):273-9 (1998)). Though 17AAG binding to Hsp90 is weaker than GA, 17AAG displays similar antitumor effects than GA and a better toxicity profile. 17AAG is currently in phase I clinical trials as an anti-tumor agent in several centers worldwide.

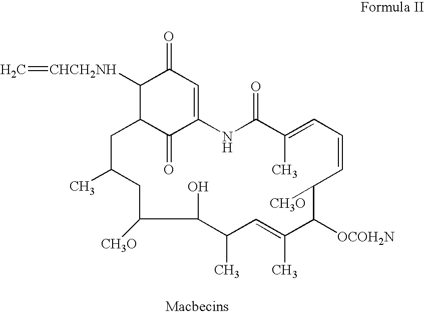

Macbecins

Macbecin I and II were isolated from the culture broth of Nocardia sp. No. C-14919. Macbecins I and II belong to the ansamycin group and have a benzoquinone and hydroquinone nucleus, respectively. (Ono et al., Gann. 73(6):938-44 (1982); Muroi et al., J. Antibiot. (Tokyo) 33(2):205-12 (1980); Tanida et al., J Antibiot (Tokyo) 33(2):199-204 (1980)).

Herbimycins

Herbimycin A is a benzochinoid ansamycin antibiotic isolated from Streptomyces sp. MH237-CF8, which specifically inhibits the phosphorylation of tyrosine residues catalyzed by various protein kinases. Omura et al., J. Antibiot. (Tokyo) 32(4):255-61 (1979). Derivatives of herbimycin have also been described, including 8,9-Epoxide, 7,9-cyclic carbamate, 17 or 19-amino derivatives, halogenated and other related derivatives; see, e.g., Shibata et al., J. Antibiot. (Tokyo) 39(3):415-23 (1986); Shibata et al., J. Antibiot. (Tokyo) 39(11):1630-3 (1986); and Oikawa et al., Biol Pharm Bull 17(10):1430-2 (1994).

Radicicol and Radicicol Analogs

Radicicol is a macrocyclic antibiotic produced by fungi, was originally isolated many years ago, and was described as tyrosine kinase inhibitor. Radicicol depletes the Hsp90 client signaling molecules in cells, and thus inhibit the signal transduction pathway. Radicicol binds directly to the N terminal ATP/ADP binding site of Hsp90. Although radicicol itself has little or no activity in animals because of instability, a number of derivatives are known and have been shown to be active, including oxime derivatives, ester derivatives, palmitoyl derivatives, and biotinylated derivatives (Soga et al., Current Cancer Drug Targets, 3(5):359-369 (2003); Ki, J. Biol. Chem., 275(50):39231-39236 (2000)).

Therapeutic Compositions and Methods of Administration

The invention includes pharmaceutical compositions including a Hsp90 inhibitor and a pharmacologically acceptable carrier; in some embodiments, the composition is specially adapted for use in the eye. As used herein, “pharmaceutically acceptable carrier” includes saline, solvents, dispersion media, coatings, antibacterial and antifungal agents, isotonic and absorption delaying agents, and the like, compatible with pharmaceutical administration. In some embodiments, the Hsp90 inhibitor may not be soluble in saline alone, and can be prepared as a suspension, or in a hydrophobic solvent.

A pharmaceutical composition is typically formulated to be compatible with its intended route of administration, e.g., intravenous, oral, intravitreal, transcleral, subtenon, subcutaneous, or topically, e.g., as an ocular ointment. Supplementary active compounds can also be incorporated into the compositions. In some embodiments, the Hsp90inhibitor is administered in conjunction with another treatment modality, e.g., a known treatment modality including a systemic or local steroid, or an immunosuppressant agent, e.g., as described herein. In some embodiments, the Hsp90 inhibitor is administrated with an agent that induces paralysis of the muscles of accommodation, e.g., cycloplegic and/or mydriatic agent. In some embodiments, the pharmaceutical composition comprising the Hsp90 inhibitor is in a form suitable for local delivery to the uveal area, e.g., an injectable or implantable form.

In some embodiments, the composition is especially adapted for administration into or around the eye. For example, a composition can be adapted to be used as eye drops, or injected into the eye, e.g., using peribulbar or intravitreal injection. Such compositions should be sterile and substantially endotoxin-free, and within an acceptable range of pH. Certain preservatives are thought not to be good for the eye, so that in some embodiments a non-preserved formulation is used. Formulation of eye medications is known in the art, see, e.g., Ocular Therapeutics and Drug Delivery: A Multi-Disciplinary Approach, Reddy, Ed. (CRC Press 1995); Kaur and Kanwar, Drug Dev. Ind. Pharm. May;28(5):473-93 (2002); Clinical Ocular Pharmacology, Bartlett et al. (Butterworth-Heinemann; 4th edition (March 15, 2001)); and Ophthalmic Drug Delivery Systems (Drugs and the Pharmaceutical Sciences: a Series of Textbooks and Monographs), Mitra (Marcel Dekker; 2nd Rev&Ex edition (Mar. 1, 2003)).

An effective amount is a dosage of the Hsp90 inhibitor sufficient to provide a medically desirable result. The effective amount will vary with the particular condition being treated, the age and physical condition of the subject being treated, the severity of the condition, the duration of the treatment, the nature of the concurrent therapy (if any), the specific route of administration and the like factors within the knowledge and expertise of the health practitioner. For example, an effective amount can depend upon the degree of severity of the uveitis. In some embodiments, the Hsp90 inhibitor are used to prevent the development or progression of uveitis, that is, they are used prophylactically in subjects at risk of developing uveitis, or in subjects that already have uveitis, but whose uveitis is likely to progress, e.g., to a more severe form of the disease. Thus, an effective amount is an amount that can lower the risk of, slow or prevent altogether the development or progression of uveitis, or can ameliorate a symptom of uveitis.

Generally, doses of Hsp90 inhibitors, e.g., geldanamycin analogs, can be from about 0.01 mg/kg per day to 1000 mg/kg per day. It is expected that doses ranging from 50-500 mg/kg will be suitable, when administered systemically; lower doses will likely be used when administered locally to the ocular or uveal area. In the event that a response in a subject is insufficient at the initial doses applied, higher doses (or effectively higher doses by a different, more localized delivery route) may be employed to the extent that patient tolerance permits. Multiple doses per day can be used to achieve appropriate systemic levels of compounds. The dosage and schedule will depend on the Hsp90 inhibitor selected; a skilled practitioner would be able to select a regimen appropriate for the particular agent and individual. A number of Hsp90 inhibitors are known in the art, e.g., as described herein, and can be used in the methods described herein.

A variety of administration routes are available. The particular mode selected will depend, of course, upon the particular drug selected, the severity of the condition being treated and the dosage required for therapeutic efficacy. The methods described herein, generally speaking, can be practiced using any mode of administration that is medically acceptable, meaning any mode that produces effective levels of the active compounds without causing clinically unacceptable adverse effects. Such modes of administration include oral, rectal, topical, nasal, transdermal, or parenteral routes. The term “parenteral” includes subcutaneous, intravenous, intraocular, intravitreal, intramuscular, or infusion. Local administration to the macular area can also be used. In some embodiments, the invention includes the use of implantable formulations, e.g., Hsp90 inhibitors such as geldanamycin analogs that are contained in a slow-release formula that can be implanted at or near the uveal area. Oral administration will typically be used for prophylactic treatment because of the convenience to the patient as well as the dosing schedule. Since geldanamycin itself is associated with serious side effects, local administration will likely be preferred when geldanamycin is used.

The delivery systems can include time-release, delayed release or sustained release delivery systems. Such systems can avoid repeated administrations of the Hsp90inhibitor, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Microcapsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono- di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which the Hsp90inhibitor is contained in a form within a matrix such as those described in U.S. Pat. Nos. 4,452,775, 4,667,014, 4,748,034 and 5,239,660 and (b) diffusional systems in which an active component permeates at a controlled rate from a polymer such as described in U.S. Pat. Nos. 3,832,253, and 3,854,480. Pump-based hardware delivery systems can be used, some of which are adapted for implantation; e.g., the Alzet® osmotic pump (DURECT Corporation, Cupertino, Calif.), which has been used to deliver therapeutic compounds to the eye. In addition, U.S. Pat. No. 6,331,313 describes a biocompatible ocular drug delivery implant device that can be used to deliver one or more Hsp90 inhibitors directly to the uveal region.

Use of a long-term sustained release implant may be particularly suitable for treatment of chronic conditions. Long-term release means that the implant is constructed and arranged to delivery therapeutic levels of the active ingredient for at least 30 days, e.g., 60 days. Long-term sustained release implants are well-known to those of ordinary skill in the art and include some of the release systems described above.

The invention further provides kits including a Hsp90 inhibitor, e.g., a geldanamycin analog, and instructions (e.g., on a label or package insert such as instructions to the patient or to the clinician) for administering the Hsp90 inhibitor to an individual in order to treat, prevent, and/or delay the development or progression of uveitis.

The ability of an Hsp90 inhibitor to treat uveitis can be confirmed using methods known in the art. Generally, the methods include administering an Hsp90 inhibitor, e.g., an Hsp90 inhibitor known in the art or identified by a method known in the art, to an animal model of uveitis. Such models are known in the art and include endotoxin-induced uveitis (EIU), e.g., in mammals including rodents (such as rats and mice), lagomorphs, or pigs. An effect on a clinically relevant parameter of uveitis is then evaluated, e.g., leukocyte adhesion (which can be evaluated in vitro with quantitative endothelial cell-neutrophil adhesion assays and ex vivo with concanavalin A lectin staining of retinal flatmounts); leukocyte activation (which can be quantified with a myeloperoxidase (MPO) activity assay); and blood-retinal barrier breakdown (which can be assessed by Evans Blue extravasation). Retinal levels of VEGF, TNF-α and leukocyte total levels of the LPS receptor CD14 can also be quantified, e.g., using an ELISA-based method; membranous CD14 levels can also be assessed, e.g., using membrane precipitations with subsequent immunoblotting. Retinal activity of NF-κB and HIF-1a can also be quantified, e.g., using a modified ELISA method as known in the art; see, e.g., Poulaki et al., Am. J. Pathol. 165(2):457-69 (2004); Poulaki et al., J. Clin. Invest. 109(6):805-15 (2002).

Additional methods for evaluating each of these effects are known in the art. For example, ability to modulate expression of a protein can be evaluated at the gene or protein level, e.g., using quantitative PCR or immunoassay methods. In some embodiments, high throughput methods, e.g., protein or gene chips as are known in the art, can be used to detect an effect on protein levels (see, e.g., Ch. 12, Genomics, in Griffiths et al., Eds. Modern genetic Analysis, 1999,W. H. Freeman and Company; Ekins and Chu, Trends in Biotechnology, 1999, 17:217-218; MacBeath and Schreiber, Science 2000, 289(5485): 1760-1763; Simpson, Proteins and Proteomics: A Laboratory Manual, Cold Spring Harbor Laboratory Press; 2002; Hardiman, Microarrays Methods and Applications: Nuts & Bolts, DNA Press, 2003).

### EXAMPLES

The invention is further described in the following examples, which do not limit the scope of the invention described in the claims.

### Example 1

### Geldanamycin Treatment Reduces the Inflammatory Changes in the LPS-Induced Uveitis Rat Model

Background: Heat-shock protein 90 (Hsp90) is the central component of a ubiquitous molecular chaperone complex that interacts with a variety of intracellular client proteins to facilitate their proper folding, prevent misfolding or aggregation, and preserve their 3-dimensional conformation to a functionally competent state.

Objective: The object of the present study was to investigate the anti-inflammatory effects of GA in endotoxin-induced uveitis (EIU) in rats.

Materials and Methods: Female Lewis rats received a single intraperitoneal injection of 1 mg/kg GA or vehicle (DMSO). ETU was induced 24 hours later by a footpad injection of 200 mg/kg lipopolysaccharide (LPS).

Twenty-four hours after the administration of LPS, leukocyte adhesion was evaluated in vitro with quantitative endothelial cell-neutrophil adhesion assays and ex vivo with concanavalin A lectin staining of retinal flatmounts, as follows. After the induction of deep anesthesia in the rat, the chest cavity was opened, and a 14-gauge perfusion cannula was introduced to the left ventricle. The right atrium was opened with a 12-gauge needle to achieve outflow. With the heart providing the motive force, 250 mL/kg PBS was administered from the perfusion cannula to remove erythrocytes and non-adherent leukocytes. Fixation was then achieved by perfusion with 1% paraformaldehyde and 0.5% glutaraldehyde at a pressure of 100 mm Hg. At this point, the heart stopped beating. A systemic blood pressure of 100 mm Hg was maintained by perfusing a total volume of 200 mL/kg over 3 minutes. Inhibition of nonspecific binding with 1% albumin in PBS (total volume 100 mL/kg) was followed by perfusion with FITC-coupled concanavalin A lectin (20 μg/mL in PBS [pH 7.4], total concentration, 5 mg/kg body weight; Vector Laboratories, Burlingame, Calif.), which stained adherent leukocytes and the vascular endothelium. Lectin staining was followed by 1% bovine serum albumin (BSA)/PBS perfusion for 1 minute, and PBS perfusion alone for 4 minutes, to remove excess concanavalin A. The retinas were flatmounted in a water-based fluorescence anti-fading medium (Fluoromount; Southern Biotechnology, Birmingham, Ala.) and imaged by fluorescence microscopy (Axioplan, FITC filter, 40×; Carl Zeiss, Oberkochen, Germany). Only whole retinas in which the peripheral collecting vessels of the ora serrata were visible were used for analysis. Leukocyte location was scored as being either arteriolar, venular, or capillary. The total number of adherent leukocytes per retina was counted. All experiments were performed in a masked fashion.

Leukocyte activation was quantified with a myeloperoxidase (MPO) activity assay and blood-retinal barrier breakdown was assessed by Evans Blue extravasation. Animals were anesthetized and Evans blue dye (Sigma) dissolved in normal saline (30 mg/ml) was injected through the tail vein at a dosage of 45 mg/kg body weight. After the dye had circulated for 120 minutes, the chest cavity was opened and blood samples were obtained from the left ventricle to assess the Evans blue plasma concentration. These blood samples were centrifuged at 16,000 g for 5 minutes. The supernatant was diluted to 1/10,000th of the initial concentration in formamide (Sigma). The absorbance of Evans blue was measured by spectrophotometry as described below. Following blood taking, the rats were perfused through the left ventricle with 20 ml citrate buffer (0.05 M, pH 3.5), 30 ml paraformaldehyde 4% in citrate buffer, and 20 ml citrate buffer at a physiological pressure of 120 mmHg. The retinas were then carefully dissected under an operating microscope. After measurement of the retinal dry weight, Evans blue was extracted by incubating each retina in 120 μl formamide for 18 hours at 70° C. The extract was filtered through a centrifugal filter tube (Ultrafree-MC 30,000 NMWL, Millipore) at 2,500 g for 2 hours at room temperature. Then, 70 μl of the extract were used for spectrophotometric measurement. The background-subtracted absorbance was determined by measuring each sample at 620 nm (absorbance maximum for Evans blue in formamide) and 740 nm (absorbance minimum). The concentrations of dye in the extracts and the blood samples were calculated using a standard curve of Evans blue in formamide. Blood-ocular barrier breakdown was calculated using the following equation, with results being expressed in microliters of plasma per gram of retina dry weight times hours of dye circulating time. Intraocular Evans blue leakage [μl /(g*h)]=(Evans blue [μg]/retina dry weight [g])*(Evans blue [μg]/plasma [μl])⁻¹*(dye circulation time [hr])⁻¹.

Retinal levels of VEGF, TNF-α, and leukocyte total levels of the LPS receptor CD14 were quantified with an ELISA method, whereas membranous CD14 levels were assessed with membrane precipitation and subsequent immunoblotting. For the ELISA, one day after the administration of the LPS rats were euthanized and retinal lysates were prepared on ice in RIPA lysis buffer (1% NP-40, 0,5% deoxycholate (DOC), 1% SDS, 150 mmol/L NaCl, 50 mmol/L Tris-HCl [pH 8.0]) containing sodium fluorid, NaVO5, phenylmethylsulfonylfluorid (PMSF), leupeptin and aprotinin. Protein concentration was determined by Bradford assay (Protein Assay, Bio-Rad, Hercules, Calif.). Samples containing the same amount of protein were assayed for their VEGF, TNF-α and CD14 levels with a commercially available kit (R&D systems, Calif.) as per the instructions of the manufacturer.

For the CD4 membranous assay, peripheral blood was obtained from diabetic and control rats anesthetized with 50 mg/kg pentobarbital via heart puncture with a 16-gauge EDTA flashed needle. Neutrophils were isolated from whole blood by density gradient centrifugation with Histopaque® 1083 cell separation media(Sigma, St Louis, Mo.) according to the manufacturer's instructions. The red blood cells were lysed with a hypotonic solution (ammonium sulphate). The preparations contained >85% monocytes as determined by eosin and methylene blue staining (Leukostat Staining System, Fisher Scientific, Pittsburgh, Pa.). Subsequently, cell surface proteins were biotinylated by incubating in 0.5 mg/ml Sulfo-NHS-LC-Biotin (Pierce, Rockford, Ill.) in PBS for 30 minutes at room temperature. Sulfo-NHS-LC-Biotin does not cross the cell membrane due to its negative charge, assuring that intracellular proteins are not biotinylated. Then, the cells were washed 3 times in cold phosphate buffered saline (PBS), scraped, centrifuged briefly and lysed for 30 min on ice in a lysis buffer (50 mM Tris-HCl, pH 8, containing 120 mM NaCl, 1% Igepal), supplemented with the Complete-TM mixture of proteinase inhibitors (Boehringer-Mannheim). The samples were cleared by centrifugation (14,000 rpm, 30 min, 40° C.) and assessed for protein concentration. Biotinylated proteins, representing the cell surface proteins, were immunoprecipitated with Streptavidin-agarose for 2 hours at 40° C., electrophoresed in an SDS-PAGE and CD14 levels were detected by Western Blotting as described.

Retinal activities of NF-κB and HIF-1α were quantified with a modified ELISA method as described in Poulaki et al., Am. J. Pathol. 165(2):457-69 (2004) and Poulaki et al., J. Clin. Invest. 109(6):805-15 (2002). NF-κB activation was analyzed using the Trans-AM NF-κB c-jun transcription factor assay kit (Active Motif, Carlsbad, Calif.) according to the manufacturer's instructions, as previously described (Mitsiades et al., Proc. Natl. Acad. Sci. USA, 99:14374-14379 (2002); Mitsiades et al., Blood, 99:4525-4530 (2002)). The nuclear extracts were incubated in 96-well plates coated with immobilized oligonucleotides containing consensus binding sites for the respective transcription factors. Transcription factor binding to the target oligonucleotide was detected by incubation with respective specific primary monoclonal antibodies, visualized by anti-IgG horseradish peroxidase conjugate and developing solution, and quantified at 450 nm with a reference wavelength of 655 nm. Background binding was calculated by adding in selected wells the respective consensus oligonucleotides in excess (20 pmol/well) as soluble competitors that prevented transcription factor binding to the probe immobilized on the plate. The resulting values were subtracted from the values obtained in wells with immobilized oligonucleotides alone. The ELISA format of this assay allowed for repeated measurements of each specimen and resulted in high sensitivity and reproducibility. For the NF-κB transcription factor assays, six animals were used from each group and retinas were not pooled.

Binding Assays for HIF-1α were performed as follows. The HIF-1α-binding site and the HIF-1α ancillary-binding site that exists in the VEGF promoter (sequence) was added at the 3′ end of a 100-bp random sequence chosen for the absence of the HIF-1 consensus sequence. The resulting 122-bp probe was produced by polymerase chain reaction using a biotinylated forward primer and for the reverse primer the HIF-1α-binding sequence. The polymerase chain reaction product was purified on ultracentrifugation membranes. The 5′ extremity of the probe is biotinylated and was linked to streptavidin-coated 96-well plates (Roche Diagnostics): 2 pmol of probe per well were incubated for 1 hour at 37° C. in 50 μl of phosphate-buffered saline (PBS). Plates were subsequently washed to remove the excess probe. Fifty μl of binding buffer were subsequently incubated with 20 μl of nuclear extracts in the wells coated with the probe, for 1 hour at room temperature with mild agitation. The wells were subsequently washed with PBS supplemented with 0.1% Tween-20 and were incubated with a monoclonal antibody against HIF-1α (Alexis Biochemicals) at a 1/1000 dilution in PBS with 1% nonfat dried milk, for 1 hour at room temperature. After the washes, the wells were incubated with a peroxidase-conjugated anti-mouse antibody (Southern Biotechnologies, Birmingham, Ala.) at a 1/1000 dilution in PBS with 1% nonfat dried milk, for 1 hour at room temperature. The peroxidase reaction was developed with tetramethylbenzidine (100 μl; Biosource, Camarillo, Calif.) that was incubated for 10 minutes at room temperature, it was stopped with 100 μl of stop solution (Biosource), and it was read at 450 nm. For the HIF-la transcription factor assay, six animals were used from each group and retinas were not pooled.

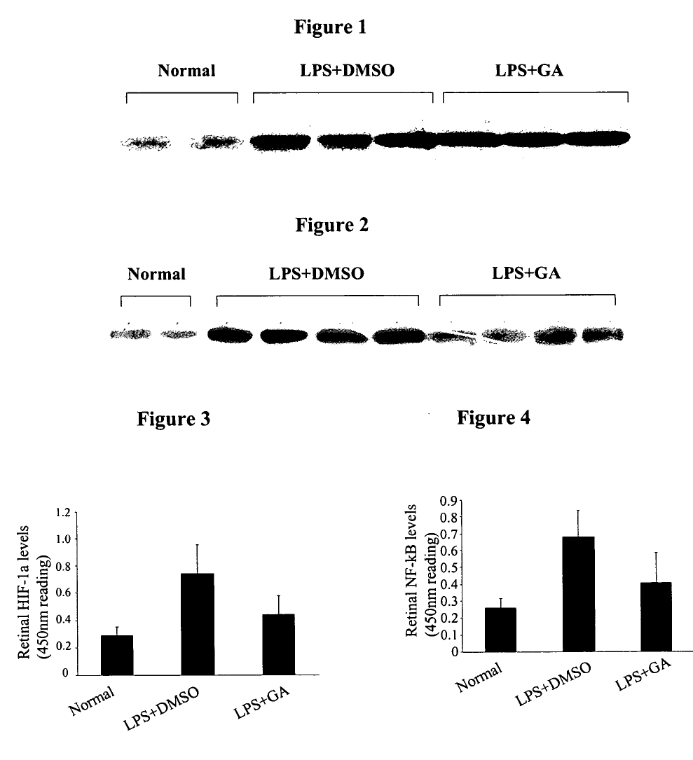

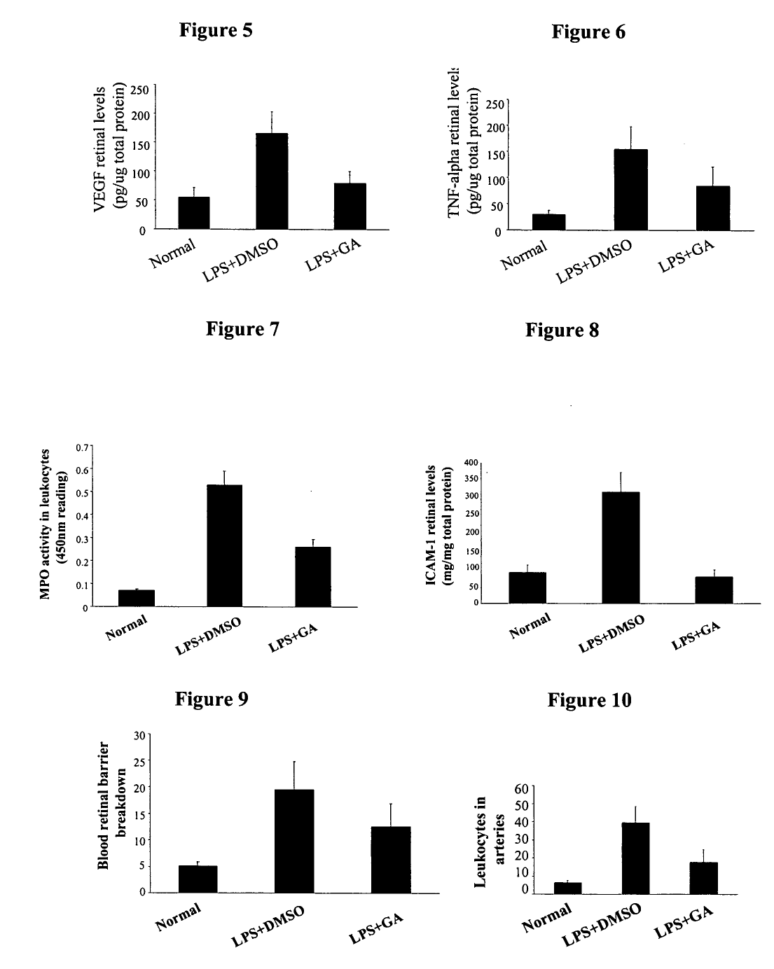

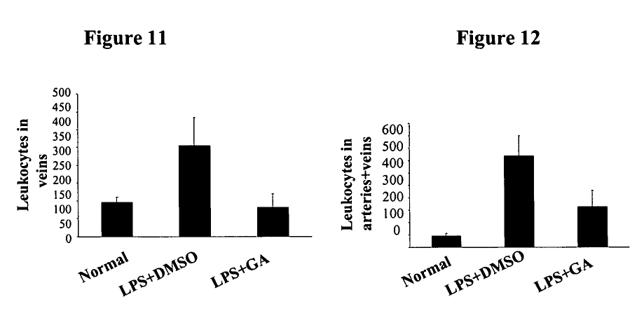

Results: Geldanamycin treatment significantly suppressed the LPS-induced increase in leukocyte adhesion both in vitro and ex vivo (see FIGS. 9-11), as well as MPO activity (see FIG. 7), vascular leakage (FIG. 12), and the LPS-induced increase of NF-κB (FIG. 4) and HIF-1a (FIG. 3) activity and VEGF (FIG. 5) and TNF-alpha (FIG. 6) levels in the retina. Although GA treatment did not reduce the LPS-induced increase in total CD14 levels in the leukocytes (FIG. 1), it significantly decreased membrane CD14 levels (FIG. 2).

Conclusions: Geldanamycin treatment suppresses the inflammatory changes in the LPS-induced uveitis model by decreasing leukocyte adhesion and activation, blood-retinal barrier breakdown and the increase in crucial proinflammatory cytokines such as VEGF and TNF-α, most likely through the observed effect on NF-κB and HIF-1a activation. Hsp90 inhibitors such as GA and its analogs, such as 17-AAG, which has demonstrated a favorable profile in phase I clinical trials in cancer patients, represent therapeutic agents for the reduction of ocular inflammation.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating uveitis in a subject, the method comprising administering to the subject a therapeutically effective amount of an inhibitor of Hsp90.

2. The method of claim 1, wherein the inhibitor of Hsp90 is a member of the ansamycin family.

3. The method of claim 1, wherein the inhibitor of Hsp90 is geldanamycin or a geldanamycin analog.

4. The method of claim 3, wherein the geldanamycin analog is 17AAG.

5. The method of claim 1, wherein the inhibitor of Hsp90 is macbecin I, macbecin II, herbimycin, or a derivative or analog thereof.

6. The method of claim 1, wherein the inhibitor of Hsp90 is radicicol or a radicicol analog.

7. The method of claim 1, wherein the inhibitor of Hsp90 is a small molecule.

8. The method of claim 7, wherein the small molecule is CCT018159 or an analog thereof.

9. The method of claim 1, wherein the inhibitor of Hsp90 is administered intravenously, orally, intravitreally, transclerally, subtenons, subcutaneously, or topically.

10. A therapeutic composition comprising an inhibitor of Hsp90, wherein the therapeutic composition is specially adapted for administration in or around the eye.

11. The therapeutic composition of claim 10, wherein the inhibitor of Hsp90 is a member of the ansamycin family.

12. The therapeutic composition of claim 10, wherein the inhibitor of Hsp90 is geldanamycin or a geldanamycin analog.

13. The therapeutic composition of claim 12, wherein the geldanamycin analog is 17AAG

14. The therapeutic composition of claim 10, wherein the inhibitor of Hsp90 is macbecin I, macbecin II, or herbimycin, or a derivative or analog thereof.

15. The therapeutic composition of claim 10, wherein the inhibitor of Hsp90 is radicicol or a radicicol analog.

16. The therapeutic composition of claim 10, wherein the inhibitor of Hsp90 is a small molecule.

17. The therapeutic composition of claim 16, wherein the small molecule is CCT018159 or an analog thereof.

18. The therapeutic composition of claim 10, wherein the therapeutic composition is adapted for administration intravitreally, transclerally, subtenons, or topically to the eye.

19. The therapeutic composition of claim 10, further comprising a carrier.
